(19) United States
(12) Patent Application Publication
(10) Pub. No.: US 2022/0093248 A1
(43) Pub. Date: Mar. 24, 2022

(54) MEDICAL CONDITION DIAGNOSIS BASED ON SEPARATE DATA SETS

(71) Applicant: International Business Machines Corporation, Armonk, NY (US)

(72) Inventors: Michael Amisano, East Northport, NY (US); Jeb R. Linton, Manassas, VA (US); David K. Wright, Monroe, MI (US); Dennis Kramer, Siler City, NC (US); John Melchionne, Kingston, NY (US); John Behnken, Hurley, NY (US)

(21) Appl. No.: 17/031,540

(22) Filed: Sep. 24, 2020

(51) Int. Cl. G16H 50/20; G16H 10/60; G16H 50/70; G16H 40/20; G06F 21/62

(52) U.S. Cl. CPC G16H 50/20; G16H 10/60; G06F 21/6245; G16H 40/20; G16H 50/70

(57) ABSTRACT

An approach for detecting potential medical conditions may be provided. Privacy laws and healthcare regulations may prevent healthcare entities from sharing data or acknowledging even seeing a patient. Secure multi-party computation can allow for the analysis of or more patient's private health data in a secure database. The private health data will only be visible to the health entity which owns or controls the data. Further, a system with oblivious random access memory may be presented which allows for the analysis of one or more patient's multiple private healthcare records. A medical condition diagnosis may be made from the analysis of the multiple private healthcare records by the secure multi-party computation using oblivious random access memory, without divulging information any private healthcare data to unauthorized parties.

### BACKGROUND

The present disclosure relates to diagnosis of medical conditions, and more specifically, to diagnosing medical conditions based on secure multi-party computation of private data.

Medical condition diagnosis may operate based on a dataset located within the care and possession of a medical provider. Patients may in some situations access medical care and treatment from multiple providers. Due to privacy laws and regulations, providers typically only have access to patient data in their possession.

### SUMMARY

According to embodiments disclosed are a method, system, and computer program product for detecting medical conditions from multi-party private health records. The approach may include sending a first patient healthcare entry for a first patient record to a first program split of a secure multi-party computation, wherein the first patient record includes one or more attributes that are related to a first patient the first patient intervention. Additionally, the approach may include detecting a medical condition, based on the first patient healthcare record at the first program split and at least one healthcare record located on at least one other program split of the secure multi-party computation. The approach may also include generating a notification that is related to the detected medical condition at the first program split. Further, the approach may include sending the notification to a first client.

The above summary is not intended to describe each illustrated embodiment or every implementation of the present disclosure.

### BRIEF DESCRIPTION OF THE DRAWINGS

The drawings included in the present application are incorporated into, and form part of, the specification. They illustrate embodiments of the present disclosure and, along with the description, serve to explain the principles of the disclosure. The drawings are only illustrative of certain embodiments and do not limit the disclosure.

FIG. 1 depicts an example Medical Condition Diagnosis Environment, consistent with some embodiments of the disclosure.

FIG. 2 depicts a method for performing patient medical condition diagnosis with private patient healthcare records from multiple parties, consistent with some embodiments of the disclosure.

FIG. 3 depicts the representative major components of an example computer system that may be operational within a Medical Condition Diagnosis Environment, in accordance with some embodiments of the present disclosure.

FIG. 4 depicts a cloud computing environment according to an embodiment of the present invention.

FIG. 5 depicts abstraction model layers according to an embodiment of the present invention.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

Aspects of the present disclosure relate to diagnosis of a medical condition, and more specifically, to diagnosis of medical conditions based on secure multi-party computation of private data from multiple parties. While the present disclosure is not necessarily limited to such applications, various aspects of the disclosure may be appreciated through a discussion of various examples using this context.

Medical condition diagnosis from private records of multiple parties (referred to as medical condition diagnosis or medical condition detection throughout this description) can be a process where healthcare providers input medical conditions into a secure database in which two or more healthcare providers maintain private healthcare records for one or more patients and a probable condition diagnosis can be made based on an analysis of multiple private medical records, while maintaining the privacy of the healthcare records. Healthcare records can be the medical history of the patient and can include diseases or infections, surgical procedures, prescriptions, therapies (psychological, physical and/or occupational), etc. . . . Private healthcare records (also referred to as private records throughout this description) are the healthcare records controlled or managed by healthcare entities such as hospitals, pharmacies, and medical clinics/practices. Medical conditions can include any physical or psychological aliment a human might encounter. Healthcare providers, such as nurses, doctors, physicians' assistants, dentists, pharmacists, and the like may operate to treat or cure a condition of the patient.

Each healthcare provider may be limited in the scope of knowledge about a patient (e.g., the information about any prior diagnosis of the patient, and the information about the patient's existing or past interventions). For example, certain laws or regulations, such as the Health Insurance Portability and Accountability Act (HIPAA) may require the safeguarding of patient data. These laws may prevent healthcare providers from disclosing, sharing, or otherwise making available any information, including patient interventions. For example, a patient may have a multitude of conditions or diagnosis. In another example, a patient may currently be prescribed a certain medication. In some examples, a patient may have exhibited a drug-seeking behavior.

This patient information and other attributes (e.g., age, name, familial relations) may be stored across many different databases controlled by various medical providers (clients). Each of the clients may or may not have the ability to share or even link this information for use by other healthcare providers. In some scenarios, healthcare providers may not be able to even acknowledge that they have seen a particular patient. This can lead to one or more of a patient's symptoms being unknown or missed resulting in undiagnosed conditions. Further, a complete view of a patient's healthcare record can be imperative for improving patient care and achieving positive expedient healthcare outcomes without creating unnecessary complications.

Consequently, a technological solution that enables the detection probable medical conditions when patient healthcare records are held in two or more private data sets may be useful. One possible solution is using a multi-party medical condition detection. For example, two healthcare providers may wish to compare information about a first patient, from two private data sources that are controlled by the two providers, respectively. Due to privacy requirements, neither of the healthcare providers may share the patient records between each other and may not consequently be able to identify any potential medical conditions for a given patient.

In some scenarios, multiple private record medical condition diagnosis techniques can only identify an exact match between two records of a patient. This may be of limited usefulness when dealing with patient records. There are many drawbacks to exact match identification. First, data is not always identical between two different healthcare providers. In many cases, data may differ. There are situations where users that are responsible for entering data may misspell attributes, values, words, or make grammatical mistakes. Sometimes, the names of individuals are spelled in an atypical fashion and the average data entry user may not enter that information properly. Sometimes, different organizations use shorthand or other abbreviations when referring to certain attributes. For example, a given intervention may have a different name or acronym depending on the healthcare provider. In another example, these acronyms may refer to the same patient, or a different patient. In some scenarios, data may be purposefully entered improperly, when individuals enter forms with partial truths or omissions. Sometimes, data in two data sets may not match because the technology fails, such as when bit rot or other data corruption occurs in one or more parties' data. Other more benign issues may occur: records that are out of date; records that have simple case, punctuation, or spacing differences. In each of these cases, private record medical condition detection techniques would not identify when two data-sets are matching.

Further, existing multi-party private record medical condition detection techniques cannot deal with more complex relationship detection between various patient records. Attributes stored about a patient may include their name, birthday, and previous addresses. Other records may also include information regarding other individuals related to the patient, such as family members. In a third example, patients may have a relationship, such as a mother and child. However, private record medical condition detection techniques may not be able to detect the relationships between the patient and other patients in these examples. A valuable record about a prescription allergy (e.g. a penicillin, anticonvulsants, chemotherapy drugs) and a familial genetic connection or mutation (e.g. BRCA1 gene, BRCA2 gene, MUTYH gene, MEN1 gene, etc. . . . ) between two patients may not be identified because multiple private record medical condition diagnosis techniques may only match patient records that are of the same patient.

A method of searching two datasets that may yield improvements is entity resolution. Entity resolution and relationship detection (entity relation operations) may be performed by a set of rules (e.g., a predetermined set of rules). The rules may function to determine when entries in two data sets refer to the same individual or refer to two individuals with a relationship. For example, a system using such a search model may decide that J. Smith and John Smith, with the same phone number, are the same person; whereas J. Smith and Alice Smith, with the same street address, are two individuals with a relationship. The drawback to this system is that—so far—private medical data may not be used in conjunction with entity-relation operations. Rather, entity-relation operations may require that the data of multiple datasets be digested, analyzed, in some cases reorganized. Further, entity-relation operations may require that evaluations are performed, and rules are validated against many, or all, other entity records. The laws and regulations around healthcare may not allow for these data sets of patient records to be shared, viewed, or otherwise intermingled—consequently, entity resolution and relationship detection may not be performed, as of yet, on patient data.

Embodiments of the disclosure may provide for medical condition detection through multi-party private healthcare record analysis by placing all of the data from two parties within a secure data store. Further, the secure data store may only be accessed in a coordinated fashion through a secure multi-party computation (SMPC) (alternatively, multi-party computation). The SMPC may operate through two or more SMPC programmatic splits. The approach to medical condition detection through multi-party private healthcare detection may function as a SMPC using one or more relevant techniques, such as Yao Construct Garbled Circuit pair. In some embodiments, SMPC may leverage the use of one or more of the following techniques: Yao Construct Garbled Circuits, Shamir Secret Sharing, Additive Secret Shares, and/or Partially Homomorphic Encryption. This may allow full featured entity resolution and relationship detection to be performed through a cooperative computation between two healthcare providers (e.g., through the programmatic splits) without requiring either organization to reveal their input data. The operations of the SMPC may provide a zero knowledge system of performing medical condition detection of various patient interventions (e.g., revealing only the absolute minimum information needed to perform a task, without leaking any other information). The operations of the SMPC may not be able to be performed without all splits. For example, an SMPC operating with two program splits may include a first split and a second split. The first split of the program splits may be unable to perform operations without the second split. Further, the second split of the program splits may be unable to perform operations without the first split. In some embodiments, the output may be revealed at the end of the computation to either or both organizations.

In some embodiments, a Three-Party Computation variant of SMPC within secure data store may occur, in which the first and second parties are organizations with an interest in detecting entity overlaps and relationships in their private data. The third party in the secure computation may be a Cloud-based Server which houses the secure data store.

In some embodiments, a Two-Party Computation variant of SMPC within a secure data store may occur. Two parties to the Two-Party Computation variant include two organizations with an interest in detecting entity overlaps and relationships in their private data. One of the two parties may agree also to host the secure data store. Security in embodiments where one party hosts the secure data store is equivalent to other embodiments through the secure data store. Specifically, the party hosting the data stored in the secure data store still cannot meaningfully inspect the data or the data access operations.

In some embodiments, computation of an SMPC may involve having three or more organizations access a common shared system which is housed in a cloud-based server. The data cooperatively stored in the secure data store may be encrypted by way of a split key. The split key may use a technique for allowing a subset of parties to access the secure data store, such as Threshold Secret Sharing. Consequently, as long as a required threshold of participants cooperates to perform multi-party computations, the SMPC can recreate the keys needed to decrypt the data in the secure storage. For example, an SMPC may be created with five splits that are each controlled by five parties, one of which may host private data for the five parties. The split key may require that four of the five parties cooperatively operate to perform entity resolution/relationship detection.

In an embodiment, a medical condition diagnosis can be determined using Bayesian inference. The inference can be made from a database of known statistical correlations between medical conditions (positive or negative). Further, the database can be comprised of oblivious random access memory (ORAM) (described further below), which keeps data secure and hidden from entities other than the client entering the data. Additionally, the database can be comprised of a large lookup table which is used to perform a Bayesian likelihood, based on new entries from clients. The large lookup table of statistical correlations can be updated based on significant correlations associated with the new entry by the client. In some embodiments, the client entering the data may receive a notification of a medical condition diagnosis, if one is detected. The patient may also receive a notification of a medical condition diagnosis, via electronic communication if a medical condition diagnosis is detected.

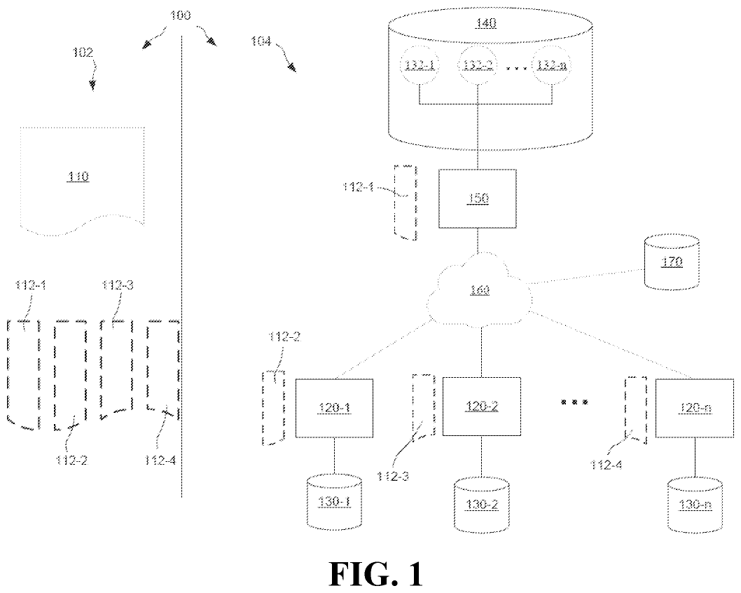

FIG. 1 depicts an example Medical Condition Diagnosis Environment (MCDE) 100, consistent with some embodiments of the disclosure. The MCDE 100 may permit analysis and enable parties to learn about relationships between records in their own private data sets and the records in other private datasets. The MCDE 100 may enable the diagnosis of previously unknown medical conditions for a patient by analysis of multiple datasets of patient records without any private data or patient records being accessed by any individual party. MCDE may leverage entity resolution and relationship detection (entity-relation operations). MCDE 100 may provide only a notice that diagnosis of a medical condition is present.

At a time 102, a program designed to perform one or more operations of environment MCDE 100 may be compiled into a program 110. During compilation, at time 102, the program 110 may be compiled into splits 112-1, 112-2, 112-3, 112-4 (collectively, 112). Each of the splits 112 may be operable by one or more clients or servers of system MCDE 100. The number of splits 112 may correspond to the number of clients and servers of a given MCDE 100. For example, in an embodiment having seven clients and one server, there may be eight splits 112 of program 110. The system MCDE 100 may operate at a time 104. Time 104 may be after time 102.

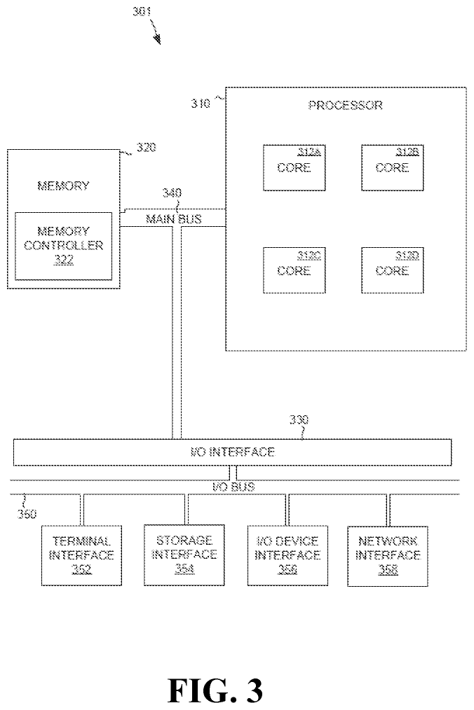

MCDE 100 may include the following: multiple clients 120-1, 120-2, to 120-n (collectively, 120); a secure data store 140; a server 150 for processing of requests to the secure data store; and a network 160 for communicatively connecting the other components of the system. Network 160 may be a network or collection of networks, including a local area network (LAN), or a wide area network, such as, the Internet. The clients 120 may be one or more computer systems or servers (and associated software) configured to receive and process requests, to host users, and to execute a split of program 110 for medical condition diagnosis. For example, FIG. 3 depicts an example computer system 301 capable of operating as a client 120 consistent with some embodiments. Each of the clients 120 may be operated or controlled by healthcare providers. For example, a first healthcare provider may own, operate, and control client 120-1, and a second healthcare provider may own, operate, and control client 120-2.

Referring back to FIG. 1, the clients 120 may each have a private data store that houses data collected and retained by a party. For example, a first party operates client 120-1 and stores and retrieves data from private data store 130-1. A second party operates client 120-2 and stores and retrieves data from private data store 130-2. Respectively, additional parties operate additional clients and store and retrieve data from other private data stores. For example, an nth party operates client 120-n and stores and retrieves data from private data store 130-n. The private data stores (collectively 130) (alternatively, other data stores) may be a database, linked list, or other data structure designed to store and retrieve records.

In some embodiments, each client 120 may be under the control of or operate under a single party. For example, a first entity affiliated with a first medical provider may be a first party fully in ownership and control of client 120-1. The first entity may own and control data as part of its normal course of operation to provide care for patients by retaining records in private data store 130-1. A second entity affiliated with one or more second additional healthcare providers may be a second party fully in ownership and control of client 120-2. The second entity may possess and/or control data as part of its normal course of operation to provide care for patients by retaining records in private data store 130-2. In such case, clients 120-1 and 120-2 (and private data stores 130-1 and 130-2, respectively) may be located geographically distant from each other.

In some embodiments, client 120-1 may not access any other data located in other data stores 130 other than data store 130-1. Likewise, client 120-2 may not access any other data located in other data stores 130 other than data store 130-2. The inability to access other records from other datastores may be due to a regulation, such as a healthcare record regulation that forbids the accessing of records held by other healthcare providers. The inability to access other records may be due to a technical reason. For example, client 120-2 may not have network connectivity to data store 130-1; client 120-2 may be technically incapable of retrieving, viewing, or otherwise accessing healthcare records of patients stored in data store 130-1.

In some embodiments, multiple parties may be assigned to operate a given client 120. For example, a client 120 may include an authentication and access management system that would enable multiple separate organizations to operate client 120. Enabling multiple separate organizations to operate client 120 may enable multi-tenancy without adding to the computational and architectural complexity of program 110. To provide for multi-tenancy, some embodiments may include distributing the same software to multiple parties and hosting multiple copies of a given client 120 (e.g., through virtual machines). In some embodiments, the distributed software may include time sharing access to a given client 120.

To ensure privacy between multiple parties in embodiments involving sharing a given client 120, data may be labeled and isolated in a given private data store 130. For example, a first party may log into client 120-2 and insert records into private data store 130-2. Upon insertion, client 120-2 may scramble, or otherwise obfuscate the records of the first party before storing those records into private data store 130-2. A second party may also log into client 120-2 (with differing credentials) and insert records into private data store 130-2. Upon insertion, client 120-2 may scramble, or otherwise obfuscate the records of the second party before storing those records into private data store 130-2. All of the records stored in private data store 130-2 may also include a tenant/owner label corresponding to each party. Client 120-2 may operate based on a relevant access control mechanism to only allow the first party and second party access only to their own records and not the records of the other.

Secure data store 140 may be a database, linked list, or other data structure designed to store and retrieve records. In some embodiments, secure data store 140 may operate such that any party cannot discern any meaning regarding the secure data store. For example, client 120-1 may be configured to host secure data store 140. Secure data store 140 may operate such that the insertion, organization, deletion, or other modification of records is opaque to inspection by client 120-1.

Secure data store 140 may utilize one or more techniques of oblivious storage. Secure data store 140 may operate in the form of Oblivious Random Access Memory (ORAM). ORAM can be thought of as a database that can run on an untrusted server, where the read and write operations are controlled by and visible to a client, but the operations are completely opaque to the server. Secure data store 140 may also operate as a working memory for hosting of one or more programs. In some embodiments, server 150, or one or more splits 112 of program 110 may be executed within secure data store 140. This may ensure that only authenticated clients have access to the operations and functioning of program 110—and the programmatic splits 112 of the program—without any party that hosts secure data store 140 able to discern any meaning of the data and operations within the secure data store.

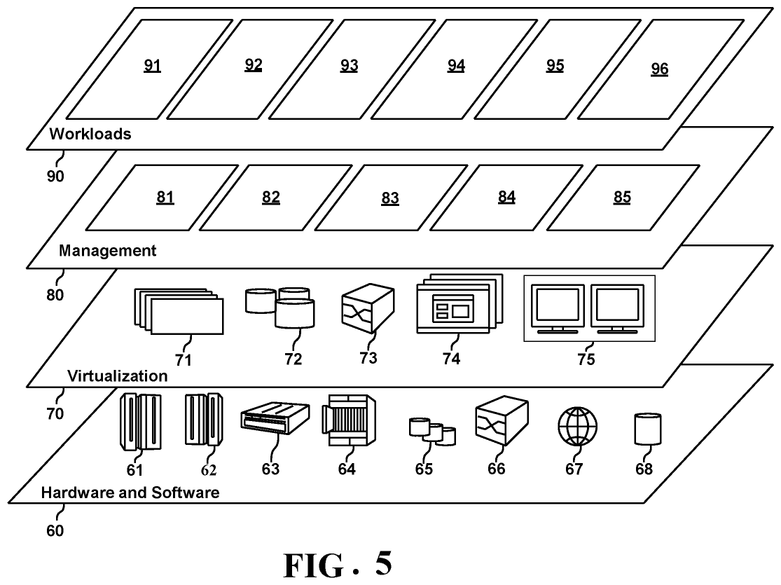

Server 150 may be a single computer system configured to perform one or more operations of system MCDE 100. For example, FIG. 3 depicts a computer system 301 operable as server 150 consistent with some embodiments. Server 150 may be operated as a service including multiple computers either alone or together. Server 150 may enable convenient, on-demand network access to a shared pool of configurable computing resources. For example, FIG. 5 depicts a series of functional abstraction layers provided by a cloud computing environment 50 capable of hosting server 150. Consequently, one or more medical condition detections may be handled by one or more layers of a cloud computing environment 50 consistent with some embodiments.

Referring back to FIG. 1, server 150 may operate by handling requests from and providing responses to clients 120 through network 160. Accordingly, server 150 may provide auditing of access by one or more of the clients. For example, server 150 may include a tracking system or ledger of activity recording all data operations of individual clients 120. Server 150 may also record all medical condition detection events, for later inspection by one or more of clients 120. Server 150 may also operate by performing data manipulation, insertion, deletion, or otherwise accessing data stored in secure data store 140. The server 150 may also connect to and access non-specific generic data 170. The generic data 170 may be stored outside of the MCDE 100, such as in a public datastore. The generic data 170 may list various drugs, drug interactions, treatments, treatment interactions, and other relevant potential medical conditions. MCDE 100 may leverage the generic data 170 to identify certain medical conditions in a given patient and existing healthcare information that may not be patient-specific.

Each client 120 may insert, view, update, or delete records it has stored within the secure data store. For example, client 120-1 may have one or more uploaded records 132-1 in secure data store 140. The uploaded records 132-1 may correspond to a subset of records in private data store 130-1. Client 120-2 may have one or more uploaded records 132-2 in secure data store 140. The uploaded records 132-2 may correspond to a subset of records in private data store 130-2. Correspondingly, client 120-n may have one or more uploaded records 132-n in secure data store 140. The uploaded records 132-n may correspond to a subset of records in private data store 130-n.

In some embodiments, insertion, viewing, updating, or deleting records may only be performed by program 110 through techniques of secure multi-party computation. Server 150 may implement secure multi-party computation to act as a sole or true client permitted to access secure data store 140 in coordination with each respective client. For example, client 120-1 may wish to access one or more records 132-1 in secure data store 140. To perform the access, split 112-2 executed by client 120-1 may operate in concert with split 112-1 executed by server 150 to perform access operations of program 110. No other program splits (e.g., 112-3, 112-4) may operate either alone or in combination to perform access operations on records 132-1; only the combination of split 112-2 and split 112-1. Likewise, records 132-2 may only be accessed by a combination of split 112-3 and split 112-1, and records 132-n may only be accessed by a combination of split 112-4 and split 112-1.

Server 150 may also implement secure multi-party computation to act as a sole or true client to perform medical condition detection, consistent with some embodiments. For example, server 150 may be embodied in the form of a garbled circuit that permits full featured entity resolution and relationship detection to be performed through a cooperative computation without revealing data inputs of the clients 120 to effectuate record medical condition detection. Entity resolution/relationship detection may be embodied in multi-party computation such that all of the splits 112-1, 112-2, 112-3, and 112-4 are required to participate in computations. In some embodiments, program 110 may be embodied such that a majority of splits 112 may operate to perform medical condition detection.

Medical condition detection within MCDE 100 may be based on entity resolution and/or relationship detection. Entity resolution may be performed based on a plurality of rules to determine if two seemingly dissimilar records are in fact the same entity. Relationship detection may be performed by a plurality of rules to determine if two seemingly similar records are actually separate but related entities. Examples of such rules include the following: Two entities with the same last name and the same address or phone number and the same birth date are a single individual. Two entities with the same last name and the same address or phone number in which one's first name is an abbreviation of the other's are a single individual, unless they have different ages, in which case they are related. Two entities with the same last name and the same address or phone number and no other shared data are related. Two individuals with the same work phone number are related. The number of rules for entity resolution/relationship detection embedded within program 110 may be, for example, between twelve and forty such rules, but could be any number.

Performing an entity-relation operation as performed by system MCDE 100 may include the process that resolves entities and detects relationships within a plurality of stored records. Each of the records may include one or more attributes, and performance of the entity resolution operation may include executing a series of concise rules against the entity received in the request. Performance of the entity-relation operation may also include execution of the rules against other records stored in a secure storage.

Performing an entity-relation operation may include processing of records in three phases: recognize, resolve, and relate. The recognition phase may include validating, optimizing, and enhancing the incoming records. During this recognize phase, the records may be cleansed and attributes may be standardized, as well as performance of data quality checks on records to protect the integrity of an entity database within a secure storage. During entity resolution, attributes within the records may be identified as entities. After the attributes in the records have been cleansed, standardized or enhanced, sophisticated search algorithms may be used to compare the attributes in the incoming record against existing entities in the entity database to determine if they are the same entity. During entity resolution, additional processing may also complete the relationship detection process, which detects relationships between identities and entities and generates alerts for relationships of interest. In some embodiments, scoring may also occur. For example, during entity resolution, it may be determined how closely attributes for an incoming record match the attributes of an existing entity. The results of this computational analysis are scores that may be used to resolve identities into entities and detect relationships between entities.

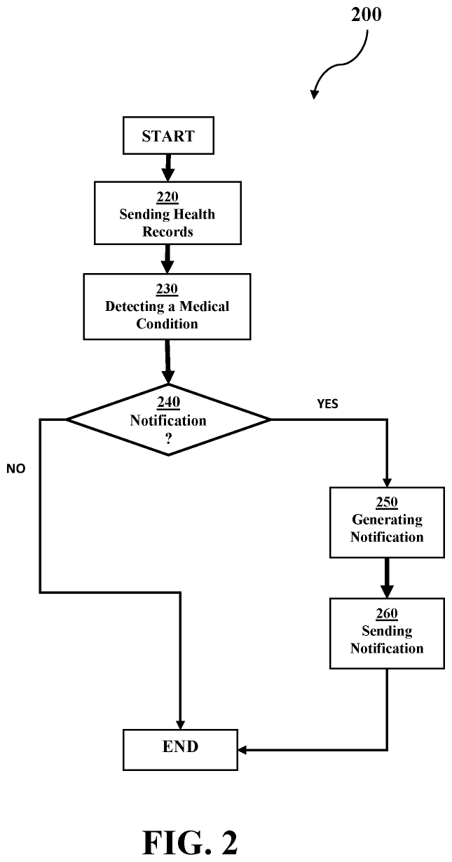

FIG. 2 depicts an example method 200 for performing medical condition detection by private patient healthcare records, consistent with some embodiments of the disclosure. Method 200 may be within by MCDE 100 as described in FIG. 1. Method 200 may be executed by a computer system, such as a server, desktop computer, or portable computing device. FIG. 3 depicts a computer system 301 operable as a computer system consistent with some embodiments. Method 200 may be provided as a service including multiple computers, either alone or together. Method 200 may be hosted as a workflow from an on-demand network access to a shared pool of configurable computing resources. FIG. 5 depicts a series of functional abstraction layers provide by a cloud computing environment 50 capable of hosting method 200 consistent with some embodiments of the disclosure. Method 200 may be performed repeatedly or continuously, such as every 100 milliseconds or every 16.6 milliseconds. In some embodiments, greater or fewer operations may be performed, or some operations may be combined or performed concurrently.

Referring back to FIG. 2, a first patient healthcare record entry may be entered at 210. The first patient healthcare record entry may be a treatment, a prescription, or symptom entered by a healthcare provider into a first patient record of the first patient. For example, a patient may have a medical record with a general practitioner of the patient. The patient may have been diagnosed with a urinary tract infection (UTI), and the general practitioner may assign the prescribe a treatment for the UTI. Other second patient records may also exist. For example, the patient may have previously visited an orthopedic physician and the orthopedic physician may have previously prescribed a different drug as a previous or existing patient intervention.

At 220 the first patient healthcare record entry may be transmitted to a first program split of an SMPC. For example, the first patient healthcare record entry may be identified by a first client. The first client may be software operated by and located at a first healthcare provider. The first client may be a remote client located in a provisioned portion of a cloud-based provider assigned to provide computing resources to the first healthcare provider. The first client may communicate with a secure data store to store, read, and modify patient records in the secure data store of the first healthcare provider. The first program split may be controlled by the first healthcare provider and may not be accessible by any other healthcare provider except for the first healthcare provider, such as through the first client.

At 230 one or more medical conditions between the first patient healthcare record entry and other patient healthcare records may be detected. Detection of one or more medical conditions can involve comparing the transmitted first patient healthcare record entry to existing patient healthcare records of other healthcare providers in a secure multi-party computation. Identification of medical conditions can involve comparing a first patient record provided as part of transmitting the first patient healthcare record entry to existent patient records in a secure multi-party computation. Identification of medical conditions can also include the comparison of the first patient intervention with a generalized (not patient specific) drug interaction data, such as a drug interaction database (stored either in the ORAM with the other patient records or imported from an outside database and compared inside of the ORAM). Identification of medical conflicts can also include the comparison of the first patient intervention with a generalized treatment and healthcare record entry data such as a medical standards and procedures database (stored either in the ORAM with the other patient records or imported from an outside database and compared inside of the ORAM). In the above example of the patient with a diagnosed UTI, the back pain could signal a possible kidney infection as well, since back pain is a symptom of kidney infection. This could result in the general practitioner performing test to check for the kidney infection.

The identification of medical conditions can leverage entity-relation operations (entity resolution and relationship detection). For example, a patient healthcare record entry may include a patient name and a symptom or test result from a medical test. A relationship detection operation may be performed to identify the patient medical records of the first client, but also of other clients in other medical systems uploaded to the ORAM by other clients. An entity resolution operation may identify that for example “Jay Jessup Smith” is the same as “J. J. Smith” based on other attributes in common (e.g., address). In another example, a first patient healthcare record entry may include a patient name, an address, and a medical complaint (e.g., pain, seeking medication). A relationship detection operation may be performed to identify relative medical records of a relative based on the patient name, the address, and another name and address of the relative medical records. For example, a patient may have recently received a mammogram which was slightly abnormal. Meanwhile, the patient's sister recently underwent genetic test which revealed she possessed the BRCA2 gene.

If a medical condition is identified (240:Y), then a notification may be generated for the first client at 250. The notification may be generated based on a privacy policy or setting of the client. The notification may be generated based on a regulation or other law that requires the consent or knowledge of the patient. In some embodiments, a first notification may be generated for the first healthcare provider that operates the first client. The first notification may remove any private information, such as by removing any information based on the first patient record. For example, the first patient record may have a series of first patient interventions and other attributes that are located within the first patient record.

The generation of the notification may be performed by removing any data or information that is not located in the first patient records. The removed data may be any information that was located in the second patient records that are in possession or control of an entity that is not the first healthcare provider or the first client. For example, the notification may be “possible of kidney infection” or “likelihood of breast cancer.” The notification may include a generic description of the diagnosis or further tests and/or treatments. For example, the notification may be “perform genetic analysis” or “perform blood culture.” In another example, the notification may be “administer IV antibiotics.” At 260, the notification may be provided to the first client such that the healthcare provider may be able to adjust the intervention based on the notification.

In some embodiments, a second notification may be generated at 250 and provided at 260 directly to the patient. For example, the notification may be “You may have a kidney infection.” The notification may be delivered to a contact attribute of the patient (e.g., an email address in the first patient record, a text to a phone number in the first patient record). The patient may then act upon the notification by electing to provide the conflict information to the healthcare provider so they can discuss an alternative intervention.

After providing the notification at 260, or after medical condition is not detected at (240:N), method 200 ends.

FIG. 3 depicts the representative major components of an example computer system 301 that may be used, in accordance with some embodiments of the present disclosure. It is appreciated that individual components may vary in complexity, number, type, and\or configuration. The particular examples disclosed are for example purposes only and are not necessarily the only such variations. The computer system 301 may comprise a processor 310, memory 320, an input/output interface (herein I/O or I/O interface) 330, and a main bus 340. The main bus 340 may provide communication pathways for the other components of the computer system 301. In some embodiments, the main bus 340 may connect to other components such as a specialized digital signal processor (not depicted).

The processor 310 of the computer system 301 may be comprised of one or more cores 312A, 312B, 312C, 312D (collectively 312). The processor 310 may additionally include one or more memory buffers or caches (not depicted) that provide temporary storage of instructions and data for the cores 312. The cores 312 may perform instructions on input provided from the caches or from the memory 320 and output the result to caches or the memory. The cores 312 may be comprised of one or more circuits configured to perform one or more methods consistent with embodiments of the present disclosure. In some embodiments, the computer system 301 may contain multiple processors 310. In some embodiments, the computer system 301 may be a single processor 310 with a singular core 312.

The memory 320 of the computer system 301 may include a memory controller 322. In some embodiments, the memory 320 may comprise a random-access semiconductor memory, storage device, or storage medium (either volatile or non-volatile) for storing data and programs. In some embodiments, the memory may be in the form of modules (e.g., dual in-line memory modules). The memory controller 322 may communicate with the processor 310, facilitating storage and retrieval of information in the memory 320. The memory controller 322 may communicate with the I/O interface 330, facilitating storage and retrieval of input or output in the memory 320.

The I/O interface 330 may comprise an I/O bus 350, a terminal interface 352, a storage interface 354, an I/O device interface 356, and a network interface 358. The I/O interface 330 may connect the main bus 340 to the I/O bus 350. The I/O interface 330 may direct instructions and data from the processor 310 and memory 320 to the various interfaces of the I/O bus 350. The I/O interface 330 may also direct instructions and data from the various interfaces of the I/O bus 350 to the processor 310 and memory 320. The various interfaces may include the terminal interface 352, the storage interface 354, the I/O device interface 356, and the network interface 358. In some embodiments, the various interfaces may include a subset of the aforementioned interfaces (e.g., an embedded computer system in an industrial application may not include the terminal interface 352 and the storage interface 354).

Logic modules throughout the computer system 301—including but not limited to the memory 320, the processor 310, and the I/O interface 330—may communicate failures and changes to one or more components to a hypervisor or operating system (not depicted). The hypervisor or the operating system may allocate the various resources available in the computer system 301 and track the location of data in memory 320 and of processes assigned to various cores 312. In embodiments that combine or rearrange elements, aspects and capabilities of the logic modules may be combined or redistributed. These variations would be apparent to one skilled in the art.

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based email). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

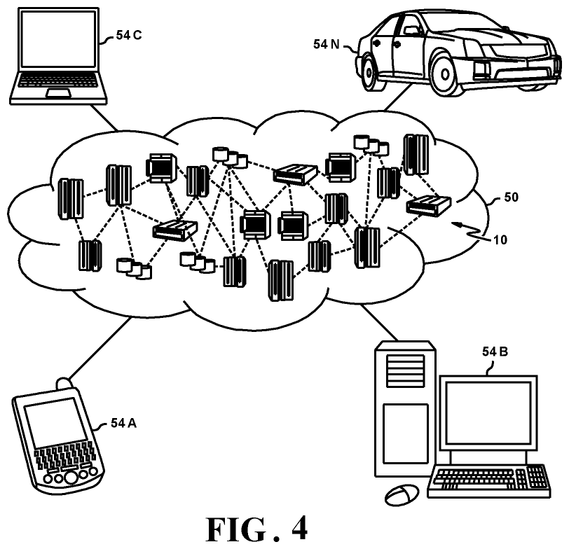

Referring now to FIG. 4, illustrative cloud computing environment 50 is depicted. As shown, cloud computing environment 50 includes one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 4 are intended to be illustrative only and that computing nodes 10 and cloud computing environment 50 can communicate with any type of computerized device over any type of network and/or network addressable connection (e.g., using a web browser).

Referring now to FIG. 5, a set of functional abstraction layers provided by cloud computing environment 50 (FIG. 4) is shown. It should be understood in advance that the components, layers, and functions shown in FIG. 5 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and secure multi party conflict detection of patient interventions 96. For example, a request to perform an entity resolution may be received by one or more clients from portal 83. The request may be passed to a first split (not depicted) of medical condition detection 96. Medical condition detection 96 may, responsively determine, without revealing any of the patient records unowned by the requester the result of the conflict detection back to management layer 80.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to explain the principles of the embodiments, the practical application or technical improvement over technologies found in the marketplace, or to enable others of ordinary skill in the art to understand the embodiments disclosed herein.

What is claimed is:

1. A computer-implemented method for detecting medical conditions from multi-party private health records comprising:
sending, by one or more processors, a first patient healthcare entry for a first patient record to a first program split of a secure multi-party computation, wherein the first patient record includes one or more attributes that are related to a first patient the first patient intervention;
detecting, by the one or more processors, a medical condition, based on the first patient healthcare record at the first program split and at least one healthcare record located on at least one other program split of the secure multi-party computation;
generating, by the one or more processors, a notification that is related to the detected medical condition at the first program split; and
sending, by the one or more processors, the notification to a first client.

2. The computer-implemented method of claim 1, wherein:
the detecting the first patient medical condition is performed by a first client;
the first client is operated by a first healthcare provider; and
the first patient record is stored in a secure data store.

3. The computer-implemented method of claim 2, wherein the first program split is only accessible by the first client.

4. The computer-implemented method of claim 2, wherein:
the detecting is based on performing an entity resolution operation;
the entity resolution operation is related to the first patient record and one or more second patient records of the first patient; and
the at least one healthcare record is uploaded to the secure data store by a second program split controlled by a second client, the second client operated by a second healthcare provider, wherein the second client is unable to access any patient records in the secure data store transmitted from the first client.

5. The computer-implemented method of claim 2, wherein:
the detecting is based on performing a relationship detection operation;
the relationship detection operation is related to the first patient record and one or more second patient records of the first patient; and
the one or more second patient records are uploaded to the secure data store by a third program split controlled by a second client, the second client operated by a second healthcare provider, wherein the second client is unable to access any patient records in the secure data store transmitted from the first client.

6. The computer-implemented method of claim 1, wherein:
the first program split is unable to perform the detecting of the conflict without the at least one additional program split; and
the at least one additional program split is not accessible by the first client.

7. The computer-implemented method of claim 1, wherein:
software is provided as a service in a cloud environment;
at least one split of the secure multi-party computation is located on a cloud computer system of a cloud computing service provider; and
a secure data store that holds the first client record is located on the cloud computer system.

8. A system for detecting medical conditions from multi-party private health records, the system comprising:
a memory, the memory containing one or more instructions; and
a processor, the processor communicatively coupled to the memory, the processor, in response to reading the one or more instructions, configured to:
send a first patient healthcare entry for a first patient record to a first program split of a secure multi-party computation, wherein the first patient record includes one or more attributes that are related to a first patient the first patient intervention;
detect a medical condition, based on the first patient healthcare record at the first program split and at least one healthcare record located on at least one other program split of the secure multi-party computation;
generate a notification that is related to the detected medical condition at the first program split; and
send the notification to a first client.

9. The system of claim 8, wherein the detecting the first patient medical condition is performed by a first client, the first client is operated by a first healthcare provider, and the first patient record is stored in a secure data store.

10. The system of claim 9, wherein the first program split is only accessible by the first client.

11. The system of claim 9, wherein the detecting is based on performing an entity resolution operation, the entity resolution operation is related to the first patient record and one or more second patient records of the first patient, and the at least one healthcare record is uploaded to the secure data store by a second program split controlled by a second client, the second client operated by a second healthcare provider, wherein the second client is unable to access any patient records in the secure data store transmitted from the first client.

12. The system of claim 8, wherein the detecting is based on performing a relationship detection operation, the relationship detection operation is related to the first patient record and one or more second patient records of the first patient, and the one or more second patient records are uploaded to the secure data store by a third program split controlled by a second client, the second client operated by a second healthcare provider, wherein the second client is unable to access any patient records in the secure data store transmitted from the first client.

13. The system of claim 8, wherein the first program split is unable to perform the detecting of the conflict without the at least one additional program split, and the at least one additional program split is not accessible by the first client.

14. The system of claim 8, wherein software is provided as a service in a cloud environment, at least one split of the secure multi-party computation is located on a cloud computer system of a cloud computing service provider, and a secure data store that holds the first client record is located on the cloud computer system.

15. A computer program product for detecting medical conditions from multi-party private health records, the computer program product comprising:
one or more computer readable storage media; and
program instructions collectively stored on the one or more computer readable storage media, the program instructions configured to:
send a first patient healthcare entry for a first patient record to a first program split of a secure multi-party computation, wherein the first patient record includes one or more attributes that are related to a first patient the first patient intervention;
detect a medical condition, based on the first patient healthcare record at the first program split and at least one healthcare record located on at least one other program split of the secure multi-party computation;
generate a notification that is related to the detected medical condition at the first program split; and
send the notification to a first client.

16. The computer program product of claim 15, wherein the detecting the first patient medical condition is performed by a first client, the first client is operated by a first healthcare provider, and the first patient record is stored in a secure data store.

17. The computer program product of claim 16, wherein the first program split is only accessible by the first client.

18. The computer program product of claim 15, wherein the detecting is based on performing an entity resolution operation, the entity resolution operation is related to the first patient record and one or more second patient records of the first patient, and the at least one healthcare record is uploaded to the secure data store by a second program split controlled by a second client, the second client operated by a second healthcare provider, wherein the second client is unable to access any patient records in the secure data store transmitted from the first client.

19. The computer program product of claim 15, wherein the detecting is based on performing a relationship detection operation, the relationship detection operation is related to the first patient record and one or more second patient records of the first patient, and the one or more second patient records are uploaded to the secure data store by a third program split controlled by a second client, the second client operated by a second healthcare provider, wherein the second client is unable to access any patient records in the secure data store transmitted from the first client.

20. The computer program product of claim 15, wherein the first program split is unable to perform the detecting of the conflict without the at least one additional program split, and the at least one additional program split is not accessible by the first client.
